(19) United States
(12) Patent Application Publication
(10) Pub. No.: US 2024/0382701 A1
(43) Pub. Date: Nov. 21, 2024

(54) INHALATION DEVICE SYSTEM WITH A COUNTING AND BLOCKING ASSEMBLY

(71) Applicant: invoX Belgium NV, Diepenbeek (BE)

(72) Inventors: Juergen Rawert, Diepenbeek (BE); Steven Dudley, Diepenbeek (BE); Amau Perdigó Oliveras, Diepenbeek (BE); Andrea Carolina Heras Briones, Diepenbeek (BE); Júlia Oliva Morell, Diepenbeek (BE); Laura Clotet Casacuberta, Diepenbeek (BE); Marc Aliana Guardia, Diepenbeek (BE)

(21) Appl. No.: 18/576,755

(22) PCT Filed: Jul. 18, 2022
(86) PCT No.: PCT/EP2022/070044

§ 371 Date: Jan. 5, 2024

(30) Foreign Application Priority Data
Jul. 20, 2021 (EP) 21186739.5
Sep. 24, 2021 (EP) 21198912.4

(51) Int. Cl. A61M 11/00; A61M 15/00

(52) U.S. Cl. CPC A61M 11/007; A61M 15/007; A61M 15/0081

(57) ABSTRACT

The present invention provides an inhalation device system for the inhalative administration of a medically active liquid in nebulized form, the system comprising an inhalation device (20) and an exchangeable reservoir (30) for holding a plurality of doses of the medically active liquid, wherein one dose of the medically active liquid is dispensed from the inhalation device per actuation of the inhalation device system, wherein the inhalation device (20) comprises—a housing (21) having a receiving unit (23), the receiving unit having a connection unit (24) adapted to releasably and fluidically connect to a connection port (31) of the exchangeable reservoir (30), the receiving unit (24) being adapted to receive and fluidically connect to the exchangeable reservoir (30); —a nozzle (25) for the nebulization of the medically active liquid; and—a pumping unit (40) arranged within the housing (21) and adapted to be fluidically connected to the exchangeable reservoir (30) and to the nozzle (25) and being adapted to convey the medically active liquid (in a downstream direction) from the exchangeable reservoir (30) to the nozzle (25) and being adapted to move the exchangeable reservoir from a resting position to a primed position upon priming of the pumping unit; wherein the inhalation device system comprises a combined counting and blocking assembly comprising a counting unit for counting the number of actuations of the inhalation device system (following the insertion of the exchangeable reservoir into the inhalation device) and a blocking unit for blocking the movement of the exchangeable reservoir from the resting position to the primed position when a defined number of actuations (following the insertion of the exchangeable reservoir into the inhalation device) is reached, wherein the counting unit and the blocking unit are physically separated from each other when the exchangeable reservoir is in the resting position and adapted to interact with each other upon each movement of the exchangeable reservoir from the resting position to the primed position.

### FIELD OF THE INVENTION

The present invention relates to the field of inhalation devices for medically active liquids. In particular, the invention relates to an inhalation device system comprising an inhalation device and an exchangeable reservoir for holding a medically active liquid, wherein the inhalation device system comprises a combined counting and blocking assembly.

### BACKGROUND OF THE INVENTION

Nebulizers or other aerosol generators for liquids have long been known from the art. Amongst others, such devices are used in medical science and therapy. There, they serve as inhalation devices for the application of active ingredients in the form of aerosols, i.e., small liquid droplets embedded in a gas. Such an inhalation device is known, e.g., from document EP 0 627 230 B1. Essential components of this inhalation device are a reservoir in which the liquid that is to be aerosolized is contained; a pumping device for generation of a pressure being sufficiently high for nebulizing the liquid; as well as an atomizing device in the form of a nozzle. By means of the pumping device, the liquid is drawn in a discrete amount, i.e., not continuously, from the reservoir and fed to the nozzle. The pumping device works without propellant and generates pressure mechanically.

A known embodiment of such an inhalation device is presented, e.g., in document WO 91/14468 A1. In such a device, the pressure in the pumping chamber which is connected to the housing is generated by movement of a moveable hollow piston. The piston is moveably arranged inside the immobile cylinder or pumping chamber. The upstream arranged inlet of the hollow piston is fluidically connected to the interior of the reservoir (i.e., reservoir pipe section). Its downstream arranged tip leads into the pumping chamber. Furthermore, a check valve that inhibits a back flow of liquid into the reservoir is arranged inside the tip of the piston.

A further inhalation device is known from WO 2018/197730 A1. The hand-held inhalation device disclosed therein comprises a housing having a user-facing side; an impingement-type nozzle for generating the nebulised aerosol by collision of at least two liquid jets, the nozzle being firmly affixed to the user-facing side of the housing such as to be immobile relative to the housing; a fluid reservoir arranged within the housing; and a pumping unit arranged within the housing, the pumping unit having an upstream end that is fluidically connected to the fluid reservoir and a downstream end that is fluidically connected to the nozzle. The pumping unit is adapted for pumping fluid from the fluid reservoir to the nozzle, and it comprises a riser pipe which is adapted to function as a piston in the pumping unit and is firmly affixed to the user-facing side of the housing such as to be immobile relative to the housing.

WO 2017/076938 A1 discloses a system with a nebulizer as well as a container with a fluid and an indicator device for such a nebulizer. A check scheme is used for indicating the number of containers already used with the nebulizer or which still can be used with the nebulizer. The indicator device indicates the number of uses performed or still possible with the current container.

WO 2019/016409 A2 discloses a nebulizer for nebulizing a liquid from a container and such a container. The nebulizer comprises a fluid pump for withdrawing the liquid in doses from the container and pressurizing the respective doses for nebulization. The container comprises an air pump with a piston/cylinder arrangement to pressurizing the liquid in the container to help withdrawing the liquid from the container. A control valve limits the air pressure acting on the liquid.

The known inhalation devices or inhalation device systems generally utilize counting and blocking systems, which usually count the number of activations of a container and block the further uses of the nebulizer until the container is replaced. This combined system is often attached to the container and exchanged together with the container, rendering the production of the container more expensive.

It is the object of the present invention to provide an inhalation system with a novel combined counting and blocking assembly which allows for more flexibility when used in inhalation devices or inhalation device systems.

### SUMMARY OF THE INVENTION

In a first aspect, the invention relates to an inhalation device system for the inhalative administration of a medically active liquid in nebulized form,
the system comprising an inhalation device (20) and an exchangeable reservoir (30) for holding a plurality of doses of the medically active liquid, wherein one dose of the medically active liquid is dispensed from the inhalation device per actuation of the inhalation device system,
wherein the inhalation device (20) comprises
a housing (21) having a receiving unit (23), the receiving unit having a connection unit (24) adapted to releasably and fluidically connect to a connection port (31) of the exchangeable reservoir (30), the receiving unit (24) being adapted to receive and fluidically connect to the exchangeable reservoir (30);
   a nozzle (25) for the nebulization of the medically active liquid; and
   a pumping unit (40) arranged within the housing (21) and adapted to be fluidically connected to the exchangeable reservoir (30) and to the nozzle (25) and being adapted to convey the medically active liquid (in a downstream direction) from the exchangeable reservoir (30) to the nozzle (25) and being adapted to move the exchangeable reservoir from a resting position to a primed position upon priming of the pumping unit;
wherein the inhalation device system comprises a combined counting and blocking assembly comprising a counting unit for counting the number of actuations of the inhalation device system (following the insertion of the exchangeable reservoir into the inhalation device) and a blocking unit for blocking the movement of the exchangeable reservoir from the resting position to the primed position when a defined number of actuations (following the insertion of the exchangeable reservoir into the inhalation device) is reached,
wherein the counting unit and the blocking unit are physically separated from each other when the exchangeable reservoir is in the resting position and adapted to interact with each other upon each movement of the exchangeable reservoir from the resting position to the primed position.

### BRIEF DESCRIPTION OF THE DRAWINGS

FIG. 1 depicts a cross-sectional view of an inhalation device system with a cartridge inserted into an inhalation device according to prior art;

FIG. 2 depicts a schematic embodiment of an inhalation device system according to the present invention;

FIG. 3 depicts a schematic view of the lower part of an inhalation device system, showing the lower part of the device with a counting unit and an exchangeable reservoir in the form of a container system comprising a blocking unit.

FIGS. 4A, 4B and 4C show an overview of an exemplary embodiment of a counting unit.

FIGS. 5A and 5B show an overview of an exemplary embodiment of a blocking unit, optionally attached to the exchangeable reservoir.

FIGS. 6A, 6B and 6C show an exemplary embodiment of the counting unit and a potential interaction with a blocking unit.

FIGS. 7A, 7B, 7C and 7D show an alternative exemplary embodiment of the counting unit.

FIGS. 8A, 8B and 8C show an exemplary embodiment of the blocking unit and the blocking mechanism.

### DETAILED DESCRIPTION OF THE INVENTION

The present invention provides an inhalation device system for the inhalative administration of a medically active liquid in nebulized form,
the system comprising an inhalation device (20) and an exchangeable reservoir (30) for holding a plurality of doses of the medically active liquid, wherein one dose of the medically active liquid is dispensed from the inhalation device per actuation of the inhalation device system,
wherein the inhalation device (20) comprises
   a housing (21) having a receiving unit (23), the receiving unit having a connection unit (24) adapted to releasably and fluidically connect to a connection port (31) of the exchangeable reservoir (30), the receiving unit (24) being adapted to receive and fluidically connect to the exchangeable reservoir (30);
   a nozzle (25) for the nebulization of the medically active liquid; and
   a pumping unit (40) arranged within the housing (21) and adapted to be fluidically connected to the exchangeable reservoir (30) and to the nozzle (25) and being adapted to convey the medically active liquid (in a downstream direction) from the exchangeable reservoir (30) to the nozzle (25) and being adapted to move the exchangeable reservoir from a resting position to a primed position upon priming of the pumping unit;
wherein the inhalation device system comprises a combined counting and blocking assembly comprising a counting unit for counting the number of actuations of the inhalation device system (following the insertion of the exchangeable reservoir into the inhalation device) and a blocking unit for blocking the movement of the exchangeable reservoir from the resting position to the primed position when a defined number of actuations (following the insertion of the exchangeable reservoir into the inhalation device) is reached,
wherein the counting unit and the blocking unit are physically separated from each other when the exchangeable reservoir is in the resting position and adapted to interact with each other upon each movement of the exchangeable reservoir from the resting position to the primed position.

The inhalation device system according to the present invention is suitable for the inhalative administration of a medically active liquid in nebulized form, wherein the term “medically active liquid” as used herein refers to a liquid compound or composition that has pharmacological activity or which comprises a compound or composition which has pharmacological activity and which is capable to improve or prevent symptoms associated with diseases, disorders or conditions, specifically of a disease, disorder or condition of the respiratory system such as pulmonary diseases, disorders or conditions in a subject, specifically in a warm-blooded animal or human, especially in a human. Specific examples of such a disease, disorder or condition comprise, but are not limited to lung diseases or conditions such as asthma and/or chronic obstructive pulmonary disease (COPD), especially COPD, or interstitial lung diseases affecting the interstitium of the lung and lung tissues such as those associated with the air passages and/or air sacs (alveoli), for example pulmonary fibrosis such idiopathic pulmonary fibrosis (IPF), interstitial pneumonias, or sarcoidosis.

Furthermore, the term “inhalative administration” as used herein refers to a route of administration in which the medically active liquid is transported to the respiratory system, specifically to the lower respiratory system such as the lungs of a subject by inhalation of a stream of air other carrier gas comprising the medically active liquid in nebulized or aerosolized form by a subject. The terms “nebulized”, “aerosolized” or “atomized” as used herein synonymously refer to a state of the medically active liquid in which it is present in the form of an aerosol having at least two phases: a continuous phase which is gaseous, such as air or another carrier gas, and which comprises a dispersed liquid phase in the form of small liquid droplets, and a liquid phase, i.e. the medically active liquid, which may itself represent a liquid solution, dispersion, suspension, or emulsion. In specific embodiments, such an aerosol has respirable particles or droplets, preferably having a mass median aerodynamic diameter (as measured by laser diffraction) of not more than about 10 μm, in particular not more than about 7 μm, or not more than about 5 μm, respectively.

In specific embodiments, the term “medically active liquid” as used herein refers to a medically active liquid in form of a pharmaceutical composition comprising at least one active pharmaceutical ingredient (API), more specifically at least one inhalable active pharmaceutical ingredient. More specifically, such at least one inhalable active pharmaceutical ingredient may, for example, be selected from long-acting muscarinic antagonists (LAMA), long-acting beta agonists (LABA) and inhalable glucocorticosteroids (ICS), as well as from analgetics and antidiabetics, either alone or in combination which each other.

Examples for long-acting muscarinic antagonists (LAMA) comprise, but are not limited to aclidinium bromide, glycopyrronium salts, such as glycopyrronium bromide, revefenacin, tiotropium, such as tiotropium bromide, umeclidinium bromide, oxitropium bromide, flutropium bromide, ipratropium bromide, trospium chloride, tolterodine.

Examples for long-acting beta agonists (LABA) comprise, but are not limited to, albuterol, arformoterol, bambuterol, bitolterol, broxaterol, carbuterol, clenbuterol, fenoterol, formoterol, hexoprenaline, ibuterol, indacaterol, indacterol, isoetharine, isoprenaline levosalbutamol, mabuterol meluadrine, metaproterenol, olodaterol, orciprenaline, pirbuterol, procaterol, reproterol, rimiterol, ritodrine, salmeterol, salmefamol, soterenot, sulphonterol, tiaramde, terbutaline, terbuterol.

Examples of inhalable glucocorticosteroids (ICS) comprise, but are not limited to, prednisolone, prednisone, butixocort propionate, flunisolide, beclomethasone, triamcinolone, budesonide, fluticasone, mometasone, ciclesonide, rofleponide, dexamethasone, etiprednol-dichloroacetat, deflazacort, etiprednol, loteprednol, RPR-106541, NS-126, ST-26.

Furthermore, active pharmaceutical ingredients may be selected from analgetics, such as opioid analgetics (e.g. morphine, fentanyl) or non-opioid analgetics (e.g. salicylic acid derivates, e.g. acetylsalicylic acid) or cannabinoids (e.g. tetrahydrocannabinol), antidiabetics, such as insulin.

The medically active liquid or liquid pharmaceutical composition that may be nebulized or aerosolized by the present inhalation device system may comprise at least one active pharmaceutically ingredient as described above but may also comprise a mixture of two or more active pharmaceutically ingredients that may be administered by inhalation.

The medically active liquid or pharmaceutical composition that may be aerosolized by the inhalation device system according to the invention is preferably formulated as a composition that is suitable, and adapted for inhalative use, in other words a composition that may be nebulized or aerosolized for inhalation and that is physiologically acceptable for inhalation by a subject.

The medically active liquid or pharmaceutical composition that may be administered by the present inhalation device system or that may by contained within the corresponding exchangeable reservoir may be in the form of a dispersion, for example a suspension with a liquid continuous phase, and a solid dispersed phase or in the form of a solution.

In further embodiments, the medically active liquid or pharmaceutical composition as described above may comprise, optionally, one or more physiologically acceptable excipients, which are suitable for inhalative use. Excipients which may be featured in the composition may include, but are not limited to, one or more buffering agents to regulate or control pH of the solution, salts, taste-masking agents, surfactants, lipids, antioxidants, and co-solvents, which may be used to enhance or improve solubility, for example ethanol, or a glycol.

In specific embodiments, the medically active liquid as described above may be essentially free of a propellant, such as a hydrofluoroalkane (HFA) propellant.

In further specific embodiments, the medically active liquid as described above may be an aqueous solution, in which one or more active pharmaceutical ingredients as described above are dissolved and solubilized in a liquid carrier solution comprising water. Such aqueous solutions optionally may also comprise one or more excipients as described above.

The inhalation device system of the present invention comprises an inhalation device and an exchangeable reservoir for holding the medically active liquid. The inhalation device of the inhalation device system of the present invention, in specific embodiments, may be a hand-held device or, in other words, a mobile device which can be conveniently held in and used with one hand and which is suitable for delivering a nebulised medically active aerosol as described above for inhalation therapy. In order to be suitable for inhalation therapy, the device must be able to emit a medically active aerosol whose particle size is respirable, i.e., small enough to be taken up by the lungs of a patient or user with respirable particles in the above-described range. In this respect, inhalation devices are substantially different from devices that emits spray for oral or nasal administration, such as disclosed in US 2004/0068222 A1.

The inhalation device of the present system comprises a housing which defines the outer casing of the inhalation device, specifically the outer casing in which the further components of the inhalation device are received and/or attached to. The housing may have a user-facing side which can be contacted by the user of the inhalation device, specifically for inhalative administration as described above. In specific embodiments, the user facing side may be a mouthpiece that may be introduced to the mouth of the user, specifically for inhalation or administration of the nebulized medically active liquid.

Furthermore, the housing may have a lower part, preferably located at the upstream end of the inhalation device, that can be moved, opened, or detached and at least partly removed to open the housing and allow access to a receiving unit into which the exchangeable reservoir can be inserted. The term “upstream” as used herein means, with regard of the present inhalation device, inhalation device system, cartridge system or other component the direction or location from which the medically active liquid is conveyed by the inhalation device during operation. In contrast to this, the term “downstream” as used herein means the opposite direction or location to which the medically active liquid is conveyed by the inhalation device during operation.

Said lower part of the housing is preferably moveable to provide access to a receiving unit as defined below. In one embodiment, this corresponds to a moveable element, which provides access to the receiving unit. In some embodiments said moveable element is permanently attached to the housing, in particular embodiments, said moveable element is connected to the housing with for example a hinge. In other embodiments, the moveable element is detachable from the housing.

In some embodiments, the housing of inhalation device has a stationary portion comprising the pumping unit, the nozzle and the receiving unit and a at least one movable portion, wherein the moveable portion is movable from a closed state and an opened state and/or from a resting position to a priming position.

The inhalation device, or more specifically, the housing of the present inhalation device comprises a receiving unit adapted to receive an exchangeable reservoir or cartridge system as described in further detail below. The receiving unit has a connection unit adapted to releasably and fluidically connect to a connection port of the exchangeable reservoir. The term “fluidically connect” as used herein means that with regards to two elements a connection, preferably a gas-tight and/or liquid-tight connection is established or may be established that allows for the transfer of the fluid such as a gas or liquid from one element to the other, preferably in a way in which such fluid is completely transferred from one element to the other.

In some embodiments a movable portion of the housing is in the form of a cap covering and closing the receiving unit of the housing.

The receiving unit of the housing is adapted to receive or, in some embodiments, to fully receive and fluidically connect to the exchangeable reservoir as described in further detail below. This means, especially with regard to the term “fully received” as used herein, that such exchangeable reservoir may be completely introduced into the receiving unit of the housing such that the receiving unit and the housing may completely enclose or encase the exchangeable reservoir, preferably in a way that when introduced into the receiving unit the surface of the exchangeable reservoir is completely enclosed by the housing of the inhalation device.

The inhalation device of the system of the present invention further comprises a nozzle for the nebulization of the medically active liquid. The person of skill in the art knows different kinds of nozzles which are suitable for the nebulization, aerosolization or atomization of the medically active liquid to be administered by the system of the present invention, such as impingement-type nozzles, swirl nozzles, orifice nozzles, surface impinging nozzles or multi-fluid nozzles. In specific embodiments, however, the nozzle of the present inhalation device is of the impingement type. This means that the nozzle is adapted to emit at least two jets of liquid which are directed such as to collide and break up into small aerosol droplets. In specific embodiments, the nozzle is firmly affixed to the housing, especially to the user-facing side of the housing of the inhalation device in such a way that it is immobile, or non-moveable, relative to the housing or at least relative to the side or part of the housing which faces the user (e.g., patient) or which, more specifically, is introduced to the mouth of a user when the device is used.

The inhalation device of the system of the present invention further comprises a pumping unit which is arranged within the housing of the inhalation device. The pumping unit is adapted to be fluidically connected to the reservoir, specifically via the connection unit of the receiving unit. In specific embodiments, the pumping unit is fluidically connected to the reservoir via the connection unit of the receiving unit. Furthermore, the pumping unit is also adapted to be fluidically connected to the nozzle or, in specific embodiments, is connected to the nozzle, and is furthermore adapted to convey or, in other words, pump the medically active liquid in a downstream direction from the reservoir to the nozzle.

The pumping unit as comprised by the inhalation device of the present invention, in specific embodiments, is suitable for and adapted to deliver the nebulised medically active liquid in a discontinuous manner, i.e., in the form of discrete units, wherein one unit is delivered per pumping cycle. In this aspect, the inhalation device differs from commonly known nebulisers such as jet nebulisers, ultrasonic nebulisers, vibrating mesh nebulisers, or electrohydrodynamic nebulisers which typically generate and deliver a nebulised aerosol continuously over a period of several seconds up to several minutes, such that the aerosol requires a number of consecutive breathing manoeuvres in order to be inhaled by the patient or user. Instead, the inhalation device of the present invention is adapted to generate and emit discrete units of aerosol, wherein each of the units corresponds to the amount (i.e., volume) of fluid (i.e., medically active liquid) which is pumped by the pumping unit in one pumping cycle into the nozzle where it is immediately aerosolised and delivered to the user or patient. Vice versa, the amount of liquid pumped by the pumping unit in one pumping cycle determines the amount of the pharmacologically active agent which the patient receives per dosing. It is therefore highly important with respect to achieving the desired therapeutic effect that the pumping unit operates precisely, reliably and reproducibly. Such inhalation devices exhibiting high precision and reproducibility, specifically incorporating a pumping unit as described in further detail below, are known to those of skill in the art and are described in WO 2018/197730 A1 the disclosure of which is incorporated herein in its entirety. It should be noted however, that the specific design of the pumping unit may be varied and that further pumping units, such as the unit described in US 2012/0090603 A1, which is incorporated herein by reference in its entirety, may also be used in the inhalation device of the present invention.

In specific embodiments, the pumping unit may be also arranged within the housing and may be adapted to function as a piston pump, also referred to as plunger pump, wherein a riser pipe functions as the piston, or plunger, which is longitudinally moveable within a hollow cylinder. The pumping unit may have an upstream end that is fluidically connected to the exchangeable reservoir and a downstream end that is fluidically connected to the nozzle. In further specific embodiments, the pumping unit may comprise a riser pipe which may be adapted to function as a piston in the pumping unit, a hollow cylinder and a lockable means for storing potential energy. The lockable means may be capable of storing potential energy when locked and may be adapted for releasing the stored energy when unlocked, such as a spiral spring or other elastic element. The lockable means may be arranged outside of and mechanically coupled to the hollow cylinder in such a way that unlocking the means results in a propulsive longitudinal movement of the cylinder towards the downstream end of the pumping unit. The inner segment of such a hollow cylinder in which the upstream end of such riser pipe moves forms a pumping chamber which has a variable volume, depending on the position of the riser pipe relative to the cylinder.

In general, if the lockable means for storing potential energy is locked and storing potential energy, the inhalation device system is in a primed state; in said primed state the reservoir is in the primed position. If the lockable means for storing potential energy is released and unlocked, the inhalation device system is in a resting state and the exchangeable reservoir is in the resting position.

The hollow cylinder which provides the pumping chamber may be fluidically connected to the exchangeable reservoir, or more specifically to the connection port of the exchangeable reservoir, either directly or indirectly, such as by means of an optional reservoir pipe (or reservoir pipe section). Similarly, the riser pipe, whose reservoir-facing, interior (upstream) end which can be received in the hollow cylinder, may be fluidically connected at its downstream or exterior end to the nozzle in a liquid-tight manner, either directly or indirectly.

In this context, the expression “hollow cylinder” refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g., with a liquid to be nebulised.

As used herein, a longitudinal movement is a movement along the main axis of the hollow cylinder, and a propulsive movement is a movement of a part in a downstream (or forward) direction.

In specific embodiments, the riser pipe of the pumping unit of the inhalation device of the invention may be arranged downstream of the cylinder and may be firmly affixed to the user-facing side of the housing such as to be immobile relative to the housing or at least to the part of the housing which comprises the user-facing side of the housing. For the avoidance of doubt, the term “firmly affixed” means either directly or indirectly (i.e., via one or more connecting parts) affixed such as to prevent relative movement between the respective parts. As the nozzle preferably is also immobile relative to the housing or the respective part of the housing, the riser pipe preferably is also immobile relative to the nozzle, and the pumping action, in these embodiments, is effected by the longitudinal movement of the hollow cylinder. A propulsive movement of the cylinder, which in this embodiment is arranged in an upstream position relative to the riser pipe, results in a decrease of the volume of the pumping chamber, and a repulsive movement of the cylinder results in an increase of the volume. In other words, in these embodiments, the riser pipe maintains its position relative to the housing, and the hollow cylinder can alter its position relative to the housing, and in particular, along a longitudinal axis of the same, such as to perform a piston-in-cylinder-type movement of the immobile riser pipe in the moveable cylindrical member.

This arrangement differs from other impingement-type inhalation devices which rely on a pumping unit whose riser pipe is in an upstream position and a cylindrical member in a downstream position wherein the riser pipe is moveable and the cylindrical member is fixed to the housing, as disclosed in US 2012/0090603 A1. A key advantage of the device having a fixed raiser pipe as described above is that the passage between pumping chamber and exchangeable reservoir can be designed with less restrictions with respect to its dimensions. It is possible to accommodate a significantly larger inlet valve (also referred to as check valve), which is easier to manufacture since it does not have to be contained within a narrow riser pipe. Instead, the fixed riser pipe design of the pumping unit allows for the use of a check valve whose size is only restricted by the interior size of the housing or the dimensions of the means for storing potential energy. In other words, the diameters of the valve, the riser pipe and—if used—the reservoir pipe do not need to match to each other. Furthermore, since in this embodiment no movable piston needs to be connected to the exchangeable reservoir, the component which provides the fluid connection to the reservoir can be designed independently of the moveable component, i.e., the hollow cylinder, allowing the individual parts to be adapted to suit their respective individual functions. In this respect, the fixed riser pipe design according to this specific embodiment provides for higher design flexibility because the moveable hollow cylinder, due to its robust structure and dimensions, provides better opportunities for designing a mechanically stable connection with the reservoir than would a less robust moveable riser pipe. Also, in this embodiment, the connection between the hollow cylinder and the exchangeable reservoir can be designed with a larger diameter, such that higher flow velocities and fluid viscosities become feasible. Further, a support for the exchangeable reservoir can be integrated into any component that comprises the cylinder. Additionally, in this embodiment, any vent for pressure equilibration of the exchangeable reservoir can be moved away from the reservoir body itself to, for example, a connector which forms an interface between exchangeable reservoir and hollow cylinder of the pumping unit, thus facilitating construction and avoiding the necessity to provide an essentially “open” reservoir body. This is especially important in cases in which the reservoir is designed as an exchangeable reservoir as it is the case in the present invention.

As mentioned above, the lockable means for storing potential energy may be adapted to store energy in its locked state and to release the stored energy when unlocked. In specific embodiments, the lockable means is mechanically coupled to the hollow cylinder in such a way such that unlocking the means results in a propulsive longitudinal movement of the hollow cylinder towards the downstream end of the pumping unit. During this movement, the internal volume of the cylinder, i.e., the volume of the pumping chamber, decreases. Vice versa, when the means for storing potential energy is in the locked state, the hollow cylinder is in its most upstream position in which the volume of the pumping chamber is largest. The locked state could also be considered a primed state. When the state of the means for storing energy is altered from the unlocked to the locked state, which could be referred to as priming the device, the hollow cylinder performs a repulsive longitudinal movement, i.e., from its most downstream position towards its most upstream position. A pumping cycle consists of two subsequent and opposing movements of the hollow cylinder starting from its most downstream position to its most upstream (or primed) position and—driven by the lockable means for storing potential energy that now releases its energy-back to its most downstream position. In preferred embodiments, the exchangeable reservoir moves together with the hollow cylinder during said movements.

In specific embodiments, the pumping unit is a high-pressure pumping unit and adapted to operate, or to expel fluid, at a pressure of at least about 50 bar. In other preferred embodiments, the operating pressure of the pumping unit is at least about 10 bar, or at least about 100 bar, or from about 2 bar to about 1000 bar, or from about 50 bar to about 250 bar, respectively. As used herein, the operating pressure is the pressure at which the pumping unit expels the medically active liquid to be administered, such as an inhalable aqueous liquid formulation of a pharmacologically active ingredient, from its pumping chamber in a downstream direction, i.e., towards the nozzle. In this context, the expression “adapted to operate” means that the components of the pumping unit are selected with respect to the materials, the dimensions, the quality of the surfaces and the finish are selected such as to enable operation at the specified pressure.

Moreover, such high-pressure pumping unit implies that the lockable means for storing potential energy is capable of storing and releasing a sufficient amount of energy to drive the propulsive longitudinal movement of the cylinder with such a force that the respective pressure is obtained.

The lockable means for the storage of potential energy may be designed as a tension or pressure spring. Alternatively, besides a metallic or plastic body, also a gaseous medium, or magnetic force utilizing material can be used as means for energy storage. By compressing or tensioning, potential energy is fed to the means. One end of the means may be supported at or in the housing at a suitable location; thus, this end is essentially immobile. With the other end, it may be connected to the hollow cylinder of the pumping unit which provides the pumping chamber; thus, this end may be essentially moveable. The means for the storage of potential energy can be locked after being loaded with a sufficient amount of energy, such that the energy can be stored until unlocking takes place. When unlocked, the means can release the potential energy (e.g., spring energy) to the cylinder with the pumping chamber, which is then driven such as to perform a (in this case, longitudinal) movement. Typically, the energy release takes place abruptly, so that a high pressure can build up inside the pumping chamber before a significant amount of medically active liquid is emitted, which results in a pressure decrease. In fact, during a significant portion of the ejection phase, an equilibrium exists of pressure delivered by the means for the storage of potential energy, and the amount of medically active liquid already emitted. Thus, the amount of medically active liquid remains essentially constant during this phase, which is a significant advantage to devices which use manual force of the user for the emission, such as the devices disclosed in documents US 2005/0039738 A1, US 2009/0216183 A1, US 2004/0068222 A1, or US 2012/0298694 A1, since manual force depends on the individual user or patient and is very likely to vary largely during the ejection phase, resulting in heterogeneous droplet formation, size, and amount. In contrast to the prior art, the means according to the invention ensures that the inhalation device delivers highly reproducible results.

In further embodiments, the means for storing potential energy may also be provided in the form of a highly pressurized gas container. By suitable arrangement and repeatable intermittent activating (i.e., opening) of the same, part of the energy which is stored inside the gas container can be released to the cylinder. This process can be repeated until the remaining energy is insufficient for once again building up a desired pressure in the pumping chamber. After this, the gas container must be refilled or exchanged.

In specific embodiments, the lockable means for storing potential energy is a spring having a load of at least 10 N in a deflected state. In preferred embodiments, the means for storing potential energy is a compression spring made of steel having a load from about 1 N to about 500 N in its deflected state. In other preferred embodiments, the compression spring from steel has a load from about 2 N to about 200 N, or from about 10 N to about 100 N, in its deflected state.

In one preferred embodiment, a single dose of the medication (i.e., of the nebulised aerosol of the medically active liquid) is contained in one unit, i.e., in the volume that is delivered from the pumping unit to the nozzle for aerosol generation in one single pumping cycle. In this case, the user or patient will prime and actuate the device only once, and inhale the released aerosol in one breathing manoeuvre, per dosing (i.e., per dosing event).

In another preferred embodiment, a single dose of the medication consists of two units of the aerosol, and thus requires two pumping cycles. Typically, the user or patient will prime the inhalation device, actuate it such as to release and inhale a unit of the aerosol, and then repeat the procedure. Alternatively, three or more aerosol units may constitute a single dosing.

The volume of fluid (e.g., of medically active liquid) that is pumped by the pumping unit of the present inhalation device system in one pumping cycle may be preferably in the range from about 0.1 μL to about 1000 μL, or from about 1 μL to about 250 μL, or from about 2 μL to about 150 μL. In particular, the volume may range from about 2 μL to about 50 μL, or from about 5 μL to about 25 μL, more specifically of from about 10 μL to about 20 μL, such as about 15 μL. These volume ranges are nearly the same as the volume of liquid phase that is contained in one unit of aerosol generated by the inhalation device, perhaps with minor differences due to minute losses of liquid in the device.

In further specific embodiments, the pumping unit of the inhalation device comprises an inlet valve, also referred to as a check valve or inlet check valve, positioned in the hollow cylinder. According to this embodiment, the interior space of the hollow cylinder, i.e., the pumping chamber, is fluidically connected with the fluid reservoir via the inlet check valve. The inlet valve allows the inflow of liquid into the pumping chamber, but prevents the backflow of medically active liquid towards, or into, the exchangeable fluid reservoir. In preferred embodiments, the position of the inlet valve may be at or near the upstream end of the cylinder such as to make nearly the entire internal volume of the hollow cylinder available for functioning as the pumping chamber. Alternatively, it may be more centrally located along the (longitudinal) main axis of the hollow cylinder such as to define an upstream segment and a downstream segment of the cylinder, the upstream segment being upstream of the inlet valve and the downstream segment being downstream of the valve. In this case, the pumping chamber is located in the downstream segment.

As mentioned, one of the advantageous effects of the specific embodiment of the pumping unit having a fixed or immobile piston as described above is that an inlet valve having relatively large dimensions may be accommodated in this position, i.e., at the upstream end of the pumping chamber. This is particularly beneficial as it allows for large dimensions of the fluid conduit(s) within the valve, thus enabling high fluid velocities which translate into a rapid filling of the pumping chamber during the priming of the inhalation device. Moreover, the use of medically active liquids having a higher viscosity than ordinary liquid formulations for inhalation, such as highly concentrated solutions of soluble active ingredients, become feasible for inhalation therapy.

In further embodiments, the inlet valve may be adapted to open only when the pressure difference between the upstream and the downstream side of the valve, i.e., the fluid reservoir side and the pumping chamber side, is above a predefined threshold value, and remains closed as long as the pressure difference is below the threshold value. In this context, the term “pressure difference” means that, irrespective of the absolute pressure values, only the relative pressure difference between the two sides is relevant for determining whether the valve blocks or opens. If, for example, the pressure on the upstream (reservoir) side is already positive (e.g., 1.01 bar due to thermal expansion), but the pressure on the downstream (pumping chamber) side is ambient pressure (e.g., 1.0 bar, no activation of the device), the pressure difference (here: 0.01 bar) is below the threshold value (e.g., 20 mbar), which allows the valve to stay closed even when subject to a positive pressure in opening direction. This means that the check valve remains closed until the threshold pressure is met, thus keeping the passage between reservoir and pumping chamber safely shut, e.g., when the inhalation device is not in use. Examples for threshold pressure differences are in the range of 1 to 1000 mbar, and more preferably between about 10 and about 500 mbar, or between about 1 and about 20 mbar.

When actuating the inhalation device of the present inhalation device system, as the means for storing potential energy alters its state from a locked state to an unlocked state, energy is released which effects the cylinder to perform its propulsive longitudinal movement, significant pressure is built up in the pumping chamber. This generates a marked pressure difference due to a high pressure in the pumping chamber and a substantially lower pressure in the fluid reservoir which exceeds the threshold value of the pressure difference, so that the check valve opens and allows the pressure chamber to become filled with medically liquid from the exchangeable reservoir.

A valve type that may be designed to operate with such a threshold pressure difference is a ball valve pre-loaded with a spring. The spring pushes the ball into its seat, and only if the pressure acting against the spring force exceeds the latter, the ball valve opens. Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of medically active liquid stored in the cartridge system during device transport, or evaporation during long-term storage of the device.

In further specific embodiments, the inhalation device of the system according to the invention further may comprise an outlet valve inside the riser pipe, or at an end of the riser pipe, for avoiding a return flow of liquid or air from the riser pipe into the hollow cylinder. In many cases, the use of such outlet valve will prove to be advantageous. Typically, the downstream end of the riser pipe is located close to the nozzle. The nozzle is in fluidic communication with the outside air. After emitting, in aerosolised form, the amount of medically active liquid which is delivered from the pumping unit through the nozzle, driven by the propulsive longitudinal movement of the cylinder, the pumping chamber must be refilled. For this purpose, it slides back on the riser pipe into its previous upstream position (i.e. performs a repulsive longitudinal movement), so that the interior volume of the pumping chamber increases. Along with this, a relative negative pressure (sometimes also referred to as “under-pressure”) is generated inside the pumping chamber which causes liquid to be sucked into the pumping chamber from the exchangeable reservoir which is located upstream of the pumping chamber. However, such relative negative pressure may also propagate downstream through the riser pipe up to the outside of the nozzle and could lead to air being sucked into the device through the nozzle, or nozzle openings, respectively. This problem can be avoided by providing an outlet valve, also referred to as outlet check valve, which opens towards the nozzle openings and blocks in the opposite direction.

Optionally, the outlet valve is of a type that blocks below (and opens above) a threshold pressure difference as described in the context of the inlet valve above. If a ball valve with a spring is used, the spring force must be directed against the pumping chamber such that when the difference between the interior pressure of the pumping chamber and the ambient pressure exceeds the threshold pressure difference value, the outlet valve opens. The advantages of such a valve correspond to the respective aforementioned advantages.

As mentioned, the outlet valve may be positioned within the riser pipe as described above. Alternatively, the inhalation device comprises an outlet valve which is not integrated within the riser pipe but positioned at or near one of the ends of the riser pipe, in particular at or near its downstream end, e.g., in a separate connector between the riser pipe and the nozzle. This embodiment may be advantageous in certain cases, e.g., if there is a need for a riser pipe with a particularly small diameter which makes the integration of a valve difficult. By accommodating the outlet valve downstream of the riser pipe, a valve with a relatively large diameter may be used, thus simplifying the requirements for the valve design.

In a further alternative embodiment, the outlet valve is absent. This embodiment may be feasible as the fluid channels of an impingement-type nozzle may have relatively small cross sections, resulting in only minor or very slow back flow of the medically active liquid at the given pressure conditions during the priming of the inhalation device. If the amount of backflow is considered acceptable in view of a particular product application, the inhaler design may be simplified by avoiding the outlet valve.

In any case, whether the inhalation device is designed with or without an outlet valve, all other options and preferences described with respect to other device features are applicable to both of these alternative embodiments.

The exchangeable reservoir for holding the medically active liquid comprised by the inhalation device system of the present invention is provided in form of a cartridge system. The cartridge system of the present invention has an overall volume Vₒ, wherein the term “overall volume” as used herein means the cubage of the entire cartridge system including all components thereof such as the outer walls of the cartridge system. The overall volume Vₒ of the entire cartridge system, in typical embodiments, may be selected within the range from about 0.1 mL to about 100 mL, or from about 0.1 mL to about 50 mL or from about 0.2 mL to about 30 mL, such as from about 2.5 mL to about 20 mL, or from about 5 mL to about 15 mL.

The exchangeable cartridge system of the present invention has an upstream end and a downstream end and comprises a container portion having an effective volume Vₑ for holding the medically active liquid and a connection port adapted to releasably and fluidically connect the cartridge system to the pumping unit, specifically via the connection unit of the receiving unit of the inhalation device.

The container portion of the present exchangeable cartridge system, in typical embodiments, has an effective volume Vₑ selected within the range of from about 0.1 to about 50 mL, or from about 0.1 mL to about 25 mL, or from about 1 mL to about 15 mL, or from about 1 to about 10 mL, specifically from about 3 mL to about 6 mL, or from about 6 mL to about 9 mL, more specifically from about 4.0 mL to about 5.0 mL or from about 7.0 mL to about 8.0 mL.

In specific embodiments, the connection port of the container portion may be in the form of a cap, such as a cap mounted on the downstream end of the container portion. The connection port may have an opening that allows for establishing a fluid connection to the inner lumen of the container portion and to the medically active liquid contained therein. The term “effective volume” means the cubage of the entire container portion including all components thereof such as the outer walls of the container portion or the connection port, such as a cap. The term “lumen” or “inner lumen” as used herein in connection with a hollow body such as the container portion or others means the inner space or cavity inside such hollow body irrespective of whether or not such inner space or cavity is completely or only partially surrounded by the outer walls of said hollow body.

In further specific embodiments, the container portion of the present exchangeable cartridge system may be in the form of a flexible container or in the form of a rigid or, in other words, dimensionally stable container. The terms “rigid” or “dimensionally stable” as used herein means that the container portion does not change its shape or volume when medically active liquid contained therein is discharged from the container during standard operation of the present inhalation device system or, in other words, when the medicinal active liquid is withdrawn from the container portion by the pumping unit during nebulization and administration of the medically active liquid. In specific embodiments of the present inhalation device system, the container portion of the present exchangeable cartridge system is in the form of a dimensionally stable container. In further specific embodiments, the container portion of the present exchangeable cartridge system is in the form of a dimensionally stable container comprising a flexible or collapsible inner container as described in further detail below, wherein the inner container contains the medically active liquid to be administered by the inhalation device system of the present invention.

Generally, the container portion, especially when provided in dimensionally stable form, may have any suitable shape that allows for the introduction of the container portion or the whole exchangeable cartridge system comprising such container portion into the inhalation device of the present inhalation device system. In specific embodiments, suitable shapes comprise but are not limited to bottle-type or tubular or cylindrical shapes, wherein symmetrical as well as non-symmetrical shapes can be implemented. Especially with regard to the axial symmetry of the container device or the entire cartridge system with regard to the main rotational axis of the container device or cartridge system connecting the center of its upstream end with the center of its downstream end, this may allow for advantageous embodiments in which the container device or cartridge system may or may not be inserted into the inhalation device in specific orientations only. In preferred embodiments, however, the inhaler device or entire cartridge system may have a substantially circular cross-sectional shape such that the container device or cartridge system may be introduced into the inhalation device independent of the rotational orientation around the longitudinal main axis.

In further embodiments, the container portion may be in the form of a bottle with a (main) opening, preferably at its downstream end, for charging or discharging the medically active liquid to be stored and administered. It should be noted, however, that the container portion may comprise further (minor) openings, e.g., for ventilation purposes.

In specific embodiments, the container portion of the exchangeable reservoir may comprise an inner container, holding the medically active liquid and having a maximum inner volume Vᵢ. The term “inner volume” (Vᵢ) as used herein in connection with the container portion means the total inner volume of the container portion that can be filled (partially or completely) with a liquid, specifically the medically active liquid to be administered by the inhalation device system according to the present invention. Accordingly, the inner volume Vᵢ of a container portion completely filled with a medically active liquid corresponds to the volume of the medically active liquid contained in such completely filled container portion. In typical embodiments, the maximum inner volume Vᵢ roughly corresponds to the effective volume Vₑ of the container portion and may be preferably selected within the range of from 0.1 to about 15 mL, or from about 1 to about 10 mL, specifically from about 3 mL to about 6 mL, or from about 6 mL to about 9 mL, more specifically from about 4.0 mL to about 5.0 mL or from about 7.0 mL to about 8.0 mL. In further embodiments, however, the maximum inner volume Vᵢ of an optional inner container may be smaller than the effective volume Vₑ of the container portion, resulting in situation in which not the entire lumen of the container portion is filled with an optional inner container.

For example, the inner container that may be contained in the container portion of the reservoir may be designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the optional inner container of the exchangeable reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established. In further embodiments, however, the inner container may have a non-flexible or rigid form wherein pressure equalization with the surrounding atmosphere during administration of the medically liquid stored therein is achieved by other means, such as inlet valves or a movable piston.

The container portion of the cartridge system, in specific embodiment may be made or manufactured from a polymeric material, specifically from a thermoplastic polymer, such as polyethylene, polypropylene, polyoxymethylene (POM), polystyrene and others. In alternative embodiments, the container portion may be made of a metal such as stainless steel, aluminum or other suitable metals or mixtures thereof. In preferred embodiments, however, the container portion is made of polyethylene or polypropylene, preferably polypropylene. It should be noted, however, that separate structures of the container portion, such as the connection port, preferably in form of a cap, may be formed from the same or another metallic or non-metallic material as described above.

In general, the cartridge system may have a symmetrical or non-symmetrical cross-sectional shape. In cases in which it might be important that the cartridge system can only be introduced or received in the receiving unit of the inhalation device in a specific orientation only, a non-symmetrical cross-section may be advantageous. On the other hand, especially to facilitate the insertion of the cartridge system, e.g., for infants or impaired users, it might be beneficial that the cartridge system has a symmetrical cross-section, such as a circular cross-section (perpendicular to the main central axis connecting the downstream end with the upstream end of the exchangeable cartridge system).

The inhalation device system of the present invention comprises a combined counting and blocking assembly comprising a counting unit for counting the number of actuations of the inhalation device system and a blocking unit for blocking the movement of the exchangeable reservoir from the resting position to the primed position when a defined number of actuations is reached. The counting unit and the blocking unit are physically separated from each other when the exchangeable reservoir is in the resting position and adapted to interact with each other upon each movement of the exchangeable reservoir from the resting position to the primed position.

In preferred embodiments, said blocking and counting assembly is operated manually and does not comprise electronic elements.

The blocking unit of the assembly prevents further use of the reservoir after a predefined number of uses. The inhalation device system may comprise further blocking mechanisms, which prevents the use of the inhalation device system under defined conditions. An example for such a blocking system is found in WO 2019/122451 A1. If a further blocking system is included in the inhalation device system, the blocking systems may work in conjunction with each other or independently of each other.

The counting and blocking assembly of the present invention is characterized by that the blocking unit and counting unit are physically separated from each other while the exchangeable reservoir is in the resting position. As a consequence, both units may be reset and/or replaced separately from each other.

Physically separated from each other in the context of the present invention means that, when the reservoir is in the resting position, no moveable part of the counting assembly is in contact with a moveable part of the blocking assembly. In some embodiments, the counting unit and blocking unit may each independently comprise a housing, which separate the two compounds. The housings of the respective units may or may not be in permanent contact and comprise openings that allow moveable parts of the blocking unit and the counting unit to interact with each other upon movement of the reservoir.

In a preferred embodiment the blocking unit and counting unit each comprise a housing, separating the two systems. Preferably, the two housings are not in contact with each other when the exchangeable reservoir is in resting position and are in contact or at least in very close proximity when the exchangeable reservoir is in the primed position.

When the inhalation device is ready for use and the reservoir is moved to the primed position, the counting assembly and the blocking assembly interact. In the context of the present invention interacting refers to at least bringing the two units in close physical proximity. In a preferred embodiment of the invention, the counting unit and blocking unit of the combined counting and blocking assembly interact with each other upon movement of the reservoir.

Said interaction may be a reciprocal interaction or a one-sided interaction. Preferably, the type of interaction is dependent on the state of the inhaler device system. For example, in one embodiment, when the exchangeable reservoir is replaced and unused, the blocking unit and counting unit may reciprocally interact and the blocking unit resets the counting unit and the counting unit initiates the blocking unit. If the number of predetermined uses for a reservoir is reached, the interaction causes a blocking mechanism of the blocking unit to activate and block further uses of the reservoir. If the reservoir is in use and the predetermined number of uses has not been reached, the blocking unit may activate the counting unit.

The result of said interaction between the blocking unit and counting unit preferably depends on the state of the inhalation device and replaceable reservoir. For example, if a new reservoir is inserted, the interaction between the counting unit and the blocking unit may resets the counting unit and/or initializes the blocking unit. After a predetermined number of uses, the interaction between the counting unit and blocking unit activates the blocking unit and blocks further use of the reservoir. In between these two states the interaction could be limited and may only comprise contacting the two units, i.e. contacting a moving part of one unit with a moving part of the other unit, or the interaction is limited to a moving a part of one unit by the other unit which is entering an opening of the housing of the unit. In some embodiments, the counting unit is resettable, preferably after exchange of the reservoir or by exchange of the reservoir. Preferably, the counting unit comprises indexing means for indexing the number of actuations of the inhalation device system and/or the remaining number of uses before replacement of the reservoir.

In a particular embodiment, the blocking unit resets or activates the counting unit after a new exchangeable reservoir is inserted upon interaction and the counting unit initiates the blocking mechanism in the blocking unit. When the device is used after the exchangeable reservoir is inserted, but the maximum number of uses has not been reached with the reservoir, the blocking unit activates the counting unit to count the number of uses during each interaction. Once the predetermined number of uses has been reached, the counting unit activates the blocking unit and prevents further use of the reservoir.

In some embodiments, the interaction of the units is achieved by movement of one of the units. In preferred embodiments, the blocking unit is moveable or moving axially or in a longitudinal movement during the priming step of the device, thereby interacting with the counting unit, while the counting unit remains in the same axial position. In some embodiments, the counting unit is moveable or moving axially or in a longitudinal movement during the priming step of the device, thereby interacting with the blocking unit.

The counting unit and the blocking unit may be firmly attached to the inhalation device system or parts thereof. The counting and blocking unit may be attached or connected to different parts of the inhalation device system. In some embodiments, one unit is attached to the housing and the other unit is attached to the exchangeable reservoir. In some embodiments one unit is reversibly and replaceable attached to the housing and the other unit is fixed to the exchangeable reservoir. In some embodiments, one unit is permanently attached to the housing or a part thereof and the other unit is permanently attached to the exchangeable reservoir and replaced with the reservoir.

In some embodiments the counting unit is attached to the housing of the inhalation device system. In particular embodiments, the counting unit is attached to the lower housing part. In some embodiments, said housing part is moveable or detachable. In some embodiments, the counting unit is removably connected to the housing part. In another embodiment the counting unit is irremovable connected to the housing part. Said housing part may be moved or detached to allow for a replacement of the exchangeable reservoir.

In some embodiments, the counting unit is attached to the inner surface of the housing of the inhalation device system. In particular embodiments, the counting unit is removably attached to the inner surface of the housing. In other embodiments, the counting unit is permanently attached to the inner surface of the housing. In some embodiments, the counting unit is attached to the inner surface of the moveable portion of the housing.

In some embodiments one unit of the counting and blocking assembly is attached to the exchangeable reservoir. In preferred embodiments, the blocking unit of the combined assembly is attached to the exchangeable reservoir. In some embodiments the blocking unit is firmly attached to the exchangeable reservoir and is replaced with the reservoir. In these embodiments the blocking unit moves together with the reservoir in the inhalation device system. In order to allow for a smooth operation of the inhalation device system it is preferable that the whole cartridge assembly does not interfere with the inhaler body. It is therefore preferable that the blocking unit has substantially the same cross-sectional diameter as the exchangeable reservoir. In some embodiments, the cross-sectional diameter is less than the cross-sectional diameter of the reservoir. It is particularly preferred that the blocking unit and the exchangeable reservoir have a circular cross-section.

The blocking unit can be attached to the exchangeable reservoir by any means. The skilled person is aware of suitable means for attachment. The blocking unit may be glued or weld to the exchangeable reservoir. Preferably, the blocking unit is attached to the outer surface of the exchangeable reservoir. In some embodiments, the blocking unit is attached to the outer surface of the exchangeable reservoir by a force- or form-fit connection.

The attachment of the blocking unit to the exchangeable reservoir may be via a housing of the blocking unit. The housing of the blocking unit may comprise an opening at the downstream end, which connects to the exchangeable reservoir. The opening of the housing of the blocking unit ideally has a diameter corresponding to the outer diameter of the upstream end of the exchangeable reservoir. Said opening may for example comprise a thread for connecting the blocking unit to the exchangeable reservoir.

The exchangeable reservoir may be in the form of an exchangeable cartridge having an upstream end and a downstream end and blocking unit is attached to the upstream end of the exchangeable reservoir.

If the blocking unit is attached to the exchangeable reservoir, it is preferred that the blocking unit and the exchangeable reservoir are assembled and sterilized prior to the filling of the reservoir with the medically active liquid.

It is preferred if the blocking unit is unable to be removed or difficult to remove from the reservoir. In preferred embodiments, wherein the blocking unit is attached to the exchangeable reservoir, the force necessary to remove the blocking unit from the exchangeable reservoir is higher than the force needed to remove the exchangeable reservoir from the receiving unit of the inhalation device. In these embodiments, the blocking unit is exchanged together with the reservoir.

In some embodiments the exchangeable reservoir is provided in the form of a cartridge having an outer (dimensionally stable) shell comprising an inner container for holding the medically active liquid in the form of a bag, specifically in the form of a collapsible bag and the blocking unit is attached to the shell.

In some embodiments, the exchangeable reservoir comprising the blocking unit has a cylindrical shape with a central longitudinal axis (A) connecting the connection port (32) of the exchangeable reservoir (31) located at the downstream end of the exchangeable reservoir (30) with the upstream end of the blocking unit attached to the upstream end of the exchangeable reservoir.

The blocking unit comprises a blocking mechanism.

In some embodiments, the blocking unit additionally comprises a housing comprising the blocking mechanism. In preferred embodiments, the blocking unit comprises a housing and a blocking mechanism, wherein the housing of the blocking unit comprises an opening for interaction between the counting unit and the blocking mechanism. In some embodiments, said opening is located at the upstream end of the blocking unit.

The blocking mechanism preferably prevents the movement of the replaceable reservoir from the rest position into the primed position after activation.

The movement may be prevented by the blocking unit itself or by interacting with other parts of the inhaler device system. In a preferred embodiment, said blocking mechanism blocks the movement of the exchangeable reservoir in connection with an activation mechanism of the counting unit.

The counting unit comprises a counting mechanism and an activation mechanism. The counting mechanism counts actuations of the inhaler device system. The term actuation of the inhaler device system refers to all steps to initiate the release of fluid. These steps include rotating the lower housing part, thereby activating a mechanism to move the reservoir into the primed position; activating an actuator member to release the reservoir from the primed position and releasing the fluid.

In the context of the present invention, the counting mechanism therefore preferably counts at least one of:
Rotation of at least one housing part;
Movement of the exchangeable reservoir;
Activations of the actuator member.

In a preferred embodiment, the counting mechanism counts an actuation as early as possible. For example, if the counting mechanism counts the rotation of the lower housing part, it is preferred that said rotation is counted early during the rotation. In a preferred embodiment, the counting mechanism is activated by a vertical movement of a counting member, which is moved by rotating the lower housing part A counting member may be part of the blocking unit.

The counting unit may comprise indicator means showing the number of uses. In some embodiments the indicator means show the number of uses of the reservoir that remain, on other embodiments, the indicator means show the number uses of the reservoir.

Alternatively, it is an option that the counting mechanism is operated by the interaction of the blocking unit with the counting unit upon movement of the exchangeable reservoir from the resting position to the primed position.

The counting unit comprises an activation mechanism in addition to the counting mechanism. In the preferred embodiments said activation mechanism interacts with the blocking unit, preferably with the blocking mechanism of the blocking unit.

The result of the interaction of the activation mechanism with the blocking unit is dependent on the state of the blocking unit and the counting unit. In preferred embodiments of the invention the interaction of the activation mechanism with the blocking unit will lead to one of the following:
If the reservoir has been replaced after a predetermined number of uses, the interaction will result in a reset of the counting unit;
If the reservoir has been in use and the predetermined number of uses is reached, the interaction will activate the blocking mechanism and prevent further use of the reservoir;
If the reservoir has been in use, and the predetermined number of uses is not yet reached, the interaction will have no effect or the interaction will activate the counting unit, counting one use of the inhalation device system.

In some embodiments the activation mechanism comprises an activation member. In preferred embodiments, the activation mechanism comprises a moveable activation member. The activation member may be a gear or a pin. In a preferred embodiment, the activation member is a pin. The activation mechanism in some embodiments is driven by the counting mechanism. In other embodiments, the activation mechanism may be driven independent of the counting mechanism.

In some embodiments the activation member of the activation mechanism interacts with the blocking mechanism and/or the counting mechanism. In some embodiments the activation member interacts with a moveable element in the blocking mechanism. In some embodiments, the activation member is a pin, which interacts after a predetermined number of uses with the blocking mechanism. For example, the activation member, after a predetermined number of uses of the reservoir, shifts a moving member in the blocking mechanism, causing the blocking mechanism to activate. In some embodiments said activation of the blocking mechanism is reversible. In preferred embodiments, said activation of the blocking mechanism is irreversible.

In a preferred embodiment, the activation member of the activation mechanism of the counting unit enters the opening of the blocking unit for interaction with the blocking mechanism upon movement of the exchangeable reservoir from the resting position to the primed position. As soon as the predefined number of uses is reached the moveable activation member activates the blocking mechanism.

In some embodiments, the moveable activation member is driven by the counting mechanism. In some embodiments, the moveable activation member is a moved gradually or step-wise by each activation of the counting mechanism. In this case, the activation member may be a pin, emerging from the counting unit, which interacts with the blocking unit upon movement of the reservoir. The pin may change position upon activation of the counting unit. As such, the position of the activation member is controlled by the counting unit.

In a preferred embodiment, the activation member is moveable and moves gradually, e.g. by stepwise movement, and the position of the member is dependent of the number of uses of the exchangeable reservoir.

In a preferred embodiment, the activation member is controlled by the counting mechanism. In some embodiments, the counting mechanism comprises a rotatable member having an, optionally circular, sloped ramp on which the activation member is supported. In a preferred embodiment, each activation of the counting mechanism moves the rotatable member on the sloped ramp, preferably the member is moved gradually by sliding on the upper surface sloped ramp.

In preferred embodiments, said rotatable member moves along the sloped surface of the ramp, preferably gradually, driven by the counting mechanism. The movement of the rotatable member also drives the activation member, which, as the rotatable member moves on the sloped surface slowly moves upwards in axial or downstream direction. When the rotatable member reaches the highest point of the slope, the activation member is in the highest position.

In a particular embodiment of the invention, the activation member is on the highest point of the sloped surface, after said predefined number of uses and/or is at the highest point when a new exchangeable reservoir is inserted. After the exchange of the reservoir, the next actuation of the inhalation device system will move the activation member to the lowest position on the sloped surface, preferably resetting the counting unit at the same time. Each actuation of the inhalation device system will then cause to move the activation member gradually, e.g. by stepwise movement, to move to the highest position on the sloped member, reaching the highest position after the predefined number of used, thereby activating the blocking mechanism of the blocking unit.

Said rotatable member and the activation member, may be in one single piece or may be two separate pieces. In a preferred embodiment said rotatable member and the activation member are one single piece or the activation member is connected to the rotatable member.

In a particular embodiment, the blocking mechanism comprises an opening for the activation member. The opening allows access for an activation member, which will be limited by a blocking element after the blocking mechanism is activated. After the predefined number of uses, the activation member would be positioned towards the upper end of the slope and preventing a larger movement of the reservoir as the space behind the opening is limited as the blocking member is preferably no longer moveable until the reservoir is exchanged allowing for a reset of the counting mechanism.

In a particular embodiment, the blocking system comprises two members, which are able to connect to each other. Said members have a moving configuration and a blocking configuration. The first member or stationary member comprises a hollow tube, in which at least a part of the second member or moving member is able to move. In the moving configuration, the second member is driven by the activation member of the counting unit during movement of the reservoir and moves inside the hollow tube of the first member. Said members may be realized as a plunger and cam. In the moving configuration the non-moving piece maintains the same relative position to the reservoir. If the activation member is moved far enough, after a predefined number of uses, the two members interlock into the blocking configuration. In blocking configuration, the both members move into a blocking position, optionally interlock, and limit the space for the activation member. In the blocking position both members are fixed and unable to move.

In an alternative embodiment, the blocking system may comprise two members, a moving member and a guiding member. The guiding member comprises a hollow tube, which allows the moving member to move. The moving member and guiding member maybe realized in the form of a plunger and cam. The movement of the moving element is driven by the activation member of the counting unit. The moving member or the guiding member may comprise a protruding element. The blocking unit optionally comprises a recess or corresponding protrusion, fitting the protruding element of one of the members. The moving element moves during the actuation of the inhaler device system, depending on the number of uses. After a predefined number of uses the protrusion of the moving or guiding element moves into said recess, thereby preventing further movement of the moving element, thereby limiting the space behind the opening to allow for only partial entry of the activation member through the opening if the housing of the blocking unit and, accordingly, to avoid full entry of the activation member.

In a further aspect, the invention relates to a cartridge system for holding a medically active liquid for nebulization and adapted for us in an inhalation device system (10) as defined above. The exchangeable cartridge system of the present invention has an upstream end and a downstream end and comprises a container portion having an effective volume Vₑ for holding the medically active liquid and a connection port adapted to releasably and fluidically connect the cartridge system to the pumping unit, specifically via the connection unit of the receiving unit of the inhalation device, wherein a blocking unit as defined above is connected to the upstream end of the container portion.

The cartridge system in general has been defined and explained in detail above. And the details above may be combined with the specific embodiments defined below.

In further specific embodiments, the container portion of the present exchangeable cartridge system is in the form of a dimensionally stable container to which a blocking unit as defined above is attached.

The blocking unit may be permanently or removably attached. Preferably, the blocking unit is permanently attached. The blocking unit is a blocking unit as defined above and preferably comprises a housing connecting to the container. The housing may be of the same or a different material than the container, the outer shell of the cartridge.

As noted above, it is preferred if the cartridge system is assembled and sterilized before the cartridge system is filled with the medical fluid.

In a further aspect, the invention relates to an inhalation device for use in the inhaler device system, wherein the inhalation device comprises
a housing having a receiving unit, the receiving unit having a connection unit adapted to releasably and fluidically connect to a connection port of the exchangeable reservoir, the receiving unit being adapted to receive and fluidically connect to the exchangeable reservoir;
a nozzle for the nebulization of the medically active liquid; and
a pumping unit arranged within the housing and adapted to be fluidically connected the nozzle and connectable to an exchangeable reservoir and being adapted to convey the medically active liquid in a downstream direction) from an exchangeable reservoir to the nozzle and being adapted to move the exchangeable reservoir from a resting position to a primed position upon priming of the pumping unit;
characterized in that the inhalation device comprises at least one of:
a counting unit as defined above, adapted to interact with a blocking unit; and/or
a blocking unit as defined above, adapted to interact with a counting unit.

In a preferred embodiment, the inhalation device comprises a counting unit as defined above-adapted to interact with a blocking unit. In a particular embodiment the blocking unit with which the counting unit interacts is part of the exchangeable reservoir. The inhalation device may comprise a further blocking device for blocking the operation, but said blocking device does not interact with the counting unit in a manner as defined above.

The invention further relates to the following numbered embodiments:
1. Inhalation device system (10) for the inhalative administration of a medically active liquid in nebulized form, the system comprising an inhalation device (20) and an exchangeable reservoir (30) for holding a plurality of doses of the medically active liquid, wherein one dose of the medically active liquid is dispensed from the inhalation device per actuation of the inhalation device system,
wherein the inhalation device (20) comprises
a housing (21) having a receiving unit (23), the receiving unit having a connection unit (24) adapted to releasably and fluidically connect to a connection port (31) of the exchangeable reservoir (30), the receiving unit (24) being adapted to receive and fluidically connect to the exchangeable reservoir (30);
a nozzle (25) for the nebulization of the medically active liquid; and
a pumping unit (40) arranged within the housing (21) and adapted to be fluidically connected to the exchangeable reservoir (30) and to the nozzle (25) and being adapted to convey the medically active liquid (in a downstream direction) from the exchangeable reservoir (30) to the nozzle (25) and being adapted to move the exchangeable reservoir from a resting position to a primed position upon priming of the pumping unit;
wherein the inhalation device system comprises a combined counting and blocking assembly comprising a counting unit for counting the number of actuations of the inhalation device system (following the insertion of the exchangeable reservoir into the inhalation device) and a blocking unit for blocking the movement of the exchangeable reservoir from the resting position to the primed position when a defined number of actuations (following the insertion of the exchangeable reservoir into the inhalation device) is reached,
wherein the counting unit and the blocking unit are physically separated from each other when the exchangeable cartridge is in the resting position and adapted to interact with each other upon each movement of the exchangeable cartridge from the resting position to the primed position.
2. Inhalation device system according to item 1, wherein the counting unit is attached to the housing of the inhalation device.
3. Inhalation device system according to item 1 or 2, wherein the blocking unit is attached to the exchangeable reservoir.
4. Inhalation device system according to any one of the preceding items, wherein the combined blocking and counting unit operates does not comprise an electronic element.
5. Inhalation device system according to any one of the preceding items, wherein the housing of inhalation device has a stationary portion (comprising the pumping unit, the nozzle and the receiving unit) and a movable portion (being movable from a closed state and an opened state and from a resting position to a priming position) and wherein the counting unit is attached to the movable portion of the housing.
6. Inhalation device system according to item 5, wherein the movable portion of the housing is in the form of a cap covering and closing the receiving unit of the housing.
7. Inhalation device system according to any one of the preceding items, wherein the counting unit is (permanently) attached to the inner surface of the (movable portion) of the housing of the inhalation device.
8. Inhalation device system according to any one of the preceding items, wherein counting unit is re-settable (upon exchange of the exchangeable reservoir).
9. Inhalation device system according to any one of the preceding items, wherein the blocking unit is firmly attached to the exchangeable reservoir.
10. Inhalation device system according to any one of items 1 to 8, wherein the blocking unit is releasably attached to the exchangeable reservoir.
11. Inhalation device system according to any one of the preceding items, wherein the exchangeable reservoir is in the form of an exchangeable cartridge having an upstream end and a downstream end and blocking unit is attached to the upstream end of the exchangeable cartridge.
12. Inhalation device system according to any one of the preceding items, wherein the blocking unit has a housing containing a blocking mechanism.
13. Inhalation device system according to item 12, wherein the housing of the blocking unit comprises an opening for physical interaction between the counting unit and the blocking mechanism.
14. Inhalation device system according to item 13, wherein the opening of the blocking unit is located at the upstream end of the blocking unit
15. Inhalation device system according to any one of the preceding items, wherein counting unit comprises a counting mechanism and an activation mechanism.
16. Inhalation device system according to any one of the preceding items, wherein the activation mechanism interacts with the blocking mechanism of the blocking unit.
17. Inhalation device system according to any one of the preceding items, wherein the activation mechanism comprises an activation member (in the form of a pin) for interaction with the blocking mechanism of the blocking unit.
18. Inhalation device system according to item 17, wherein the activation member of the counting unit enters the opening of the blocking unit for interaction with the blocking mechanism upon movement of the exchangeable reservoir from the resting position to the primed position.
19. Inhalation device system according to item 17 or 18, wherein the position of the activation member is controlled by the counting mechanism.
20. Inhalation device system according to any one of the preceding items, wherein the counting mechanism is operated by (physical/mechanical) interaction with the blocking unit upon movement of the exchangeable reservoir from the resting position to the primed position.
21. Inhalation device system according to any one of the preceding items, wherein the counting mechanism comprises indexing means for indexing the number of actuations of the inhalation device system (following the insertion of the exchangeable cartridge).
22. Inhalation device system according to any one of the preceding items, wherein the counting mechanism comprises a rotatable member having a (circular) sloped ramp on which the activation member is supported.
23. Inhalation device system according to any one of the preceding items, wherein the blocking unit comprises a blocking member which is initially activated to be able to “transferred” into a blocking state upon interaction with the counting mechanism after a defined number of actuations of the inhalation device system (following the insertion of the exchangeable cartridge).
24. Inhalation device system according to any one of the preceding items, wherein the blocking member of the blocking unit is moved into a blocking position when a defined number of actuations (following the insertion of the exchangeable reservoir into the inhalation device) is reached.
25. The inhalation device system (10) according to any one of the preceding items, wherein the exchangeable reservoir is provided in the form of a cartridge having an outer (dimensionally stable) shell comprising an inner container for holding the medically active liquid in the form of a bag, specifically in the form of a collapsible bag.
26. The inhalation device system (10) according to any one of items 10 to 25, wherein the force necessary to remove the blocking unit from the exchangeable reservoir is higher than the force needed to remove the exchangeable reservoir from the receiving unit (23) of the inhalation device (20).
27. The inhalation device system (10) according to any one of the preceding items, wherein the exchangeable reservoir comprising the blocking unit has a cylindrical shape with a central longitudinal axis (A) connecting the connection port (32) of the exchangeable reservoir (31) located at the downstream end of the exchangeable reservoir (30) with the upstream end of the blocking unit attached to the upstream end of the exchangeable reservoir.
28. The inhalation device system (10) according to any one of the preceding items, wherein the exchangeable reservoir (31) has an upstream end (34) and a downstream end (35) and wherein the connection port (32) is located at the downstream end (34) of the exchangeable reservoir (31) and wherein the extension the blocking unit is attached to the upstream end (35) of the exchangeable reservoir.
29. The inhalation device system (10) according to any one of the preceding items, wherein the blocking unit (33) is attached to the outer surface of the exchangeable reservoir (31) by a force- or form-fit connection.
30. The inhalation device system (10) according to any one of the preceding items, wherein the blocking unit (33) has substantially the same cross-sectional diameter as the exchangeable reservoir (31).
31. The inhalation device system (10) according to any one of the preceding items, wherein the blocking unit (33) and the exchangeable reservoir (30) have a circular cross-section.
32. The inhalation device system (10) according to any one of the preceding items, wherein the (housing of the) blocking unit (33) has an opening (37) at the downstream end, the opening having a diameter corresponding to the (outer) diameter of the upstream end (34) of the exchangeable reservoir.
33. The inhalation device system (10) according to any one of the preceding items, wherein the exchangeable reservoir (31) and the blocking unit (33) are assembled and sterilized prior to the filling of the cartridge system (30) with the medically active liquid.
34. The inhalation device system (10) according to any one of the preceding items, wherein the exchangeable reservoir (30) has an effective volume Vₑ selected within the range of from about 0.1 to about 15 mL.
35. The inhalation device system (10) according to any one of the preceding items, wherein the pumping unit (40) of the inhalation device (20) comprises an upstream end that is fluidically connected to the exchangeable reservoir (30);
   a downstream end that is fluidically connected to the nozzle (25);
wherein the pumping unit further comprises
   (i) a riser pipe (43) having an upstream end, wherein the riser pipe (43) is
      adapted to function as a piston in the pumping unit, and
      firmly affixed to the user-facing side of the housing (21) such as to be immobile relative to the housing (21); and
   (ii) a hollow cylinder (41) located upstream of the riser pipe (44), wherein the upstream end of the riser pipe (43) is inserted in the cylinder (41) such that the cylinder (41) is longitudinally movable on the riser pipe (43).
36. The inhalation device system (10) according to item 35, wherein the pumping unit (40) comprises
   (iii) a lockable means for storing potential energy (46) when locked and for releasing the stored energy when unlocked, the means (46) being arranged outside of, and mechanically coupled to, the cylinder (41) such that unlocking the means (46) results in a propulsive longitudinal movement of the cylinder (41) towards the downstream end of the pumping unit.
37 The inhalation device system according to any one of the preceding items, wherein the inhalation device is a hand-held inhalation device.
38. The inhalation device system according to any one of the preceding items, wherein the inhalation device is a soft-mist inhaler comprising at least one impingement-type nozzle.
39. The inhalation device system according to item 38, wherein the at least one impingement-type nozzle comprises at least two channels for the ejection of at least two jets of the medically active liquid, wherein the at least two liquid channels are oriented such that the trajectories of the at least two jets intersect in at least one collision point.
40. The inhalation device system according to any one of the preceding items, wherein the inhalation device system and/or the medically active liquid does not comprise a propellant, such as a hydrofluorocarbon (HFC) propellant.
41. An exchangeable cartridge system (30) for holding a medically active liquid for nebulization and adapted for us in an inhalation device system (10) according to any one of the preceding items, wherein the cartridge system comprises a container portion having an effective volume Vₑ for holding the medically active liquid and a connection port adapted to releasably and fluidically connect the cartridge system to the pumping unit, specifically via the connection unit of the receiving unit of the inhalation device, wherein a blocking unit is connected to the upstream end of the container portion.

### DETAILED DESCRIPTION OF THE DRAWINGS

The invention is further described with reference to the drawings. The figures show specific but non-limiting embodiments of the invention.

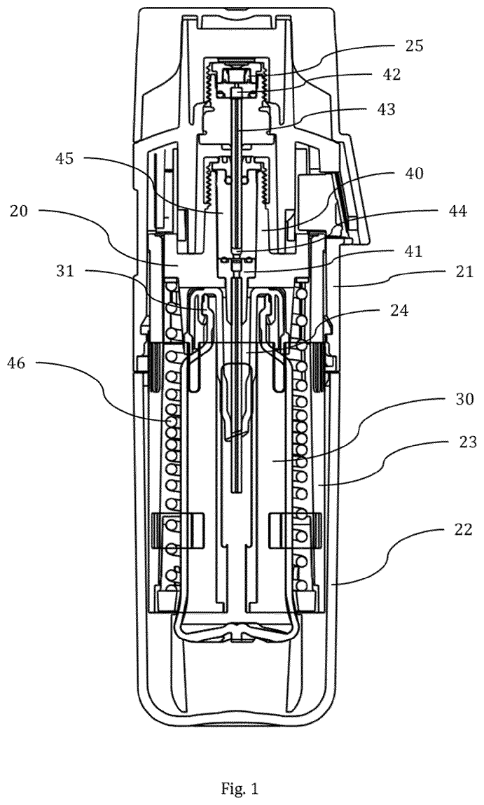

FIG. 1 shows an inhalation device system (10) according to the prior art comprising an inhalation device (20) and an exchangeable reservoir in the form of a cartridge system (30) (wherein the different elements of the cartridge system are not depicted) inserted into the inhalation device. The inhalation device (20) has a housing (21) with a lower part (22) that can be detached from the inhalation device (20) and removed to open the housing (21) and allow access to the receiving unit (23) in which the exchangeable cartridge system (30) can be inserted. The receiving unit (23) further has a connection unit (24) adapted to releasably and fluidically connect to a connection port (32) of the exchangeable reservoir.

The inhalation device (20) further has a nozzle (25) located at the downstream end of the inhalation device for nebulization of the medically active liquid. The inhalation device further has a pumping unit (40) which is arranged within the housing (21). As described in detail above, the pumping unit is fluidically connected to the reservoir (via the connection unit (24) of the receiving unit (23)) and to the nozzle (25) and is adapted to pump the medically active liquid in a downstream direction from the reservoir (30) to the nozzle (25).

The pumping unit (40) has an upstream end (41) that is fluidically connected to the exchangeable reservoir (30); a downstream end (42) that is fluidically connected to the nozzle (25); wherein the pumping unit (40) further comprises (i) a riser pipe (43) having an upstream end (44), wherein the riser pipe (43) is adapted to function as a piston in the pumping unit (40), and wherein the riser pipe (43) is firmly affixed to the user-facing (downstream) side of the housing (21) such as to be immobile relative to the housing (21); and (ii) a hollow cylinder (45) located upstream of the riser pipe (43), wherein the upstream end of the riser pipe (44) is inserted in the cylinder (45) such that the cylinder (45) is longitudinally movable on the riser pipe (43).

As also shown in FIG. 1, the pumping unit (40) comprises (iii) a lockable means for storing potential energy (46) when locked and for releasing the stored energy when unlocked, the means (46) being arranged outside of, and mechanically coupled to, the cylinder (45) such that unlocking the means (46) results in a propulsive longitudinal movement of the cylinder (45) towards the downstream end of the pumping unit (42)

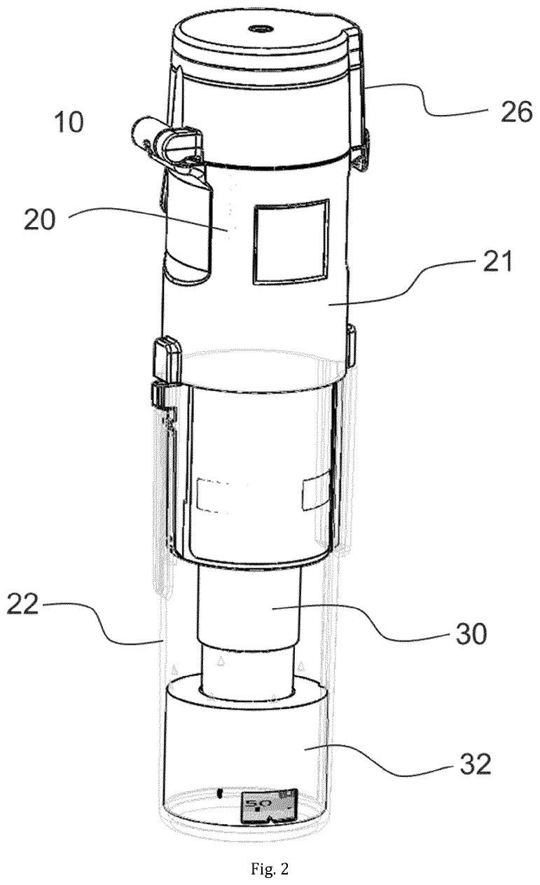

FIG. 2 shows an exemplary embodiment of an inhalation device system (10) according to the present invention, in a fully assembled state. The inhalation device system comprises an inhalation device (20) with an inserted exchangeable reservoir in the form of a cartridge system (30). In this particular embodiment, the inhalation device comprises a counting unit (32) while the cartridge system of the reservoir comprises a blocking unit (33)

The counting unit is part of the lower housing portion (22) of the inhalation device (20). The lower housing portion (22) can be removably detached or opened, for example by a hinge (not shown).

The cartridge system (30) comprises a blocking unit (33), adapted to interact with a counting unit (32).

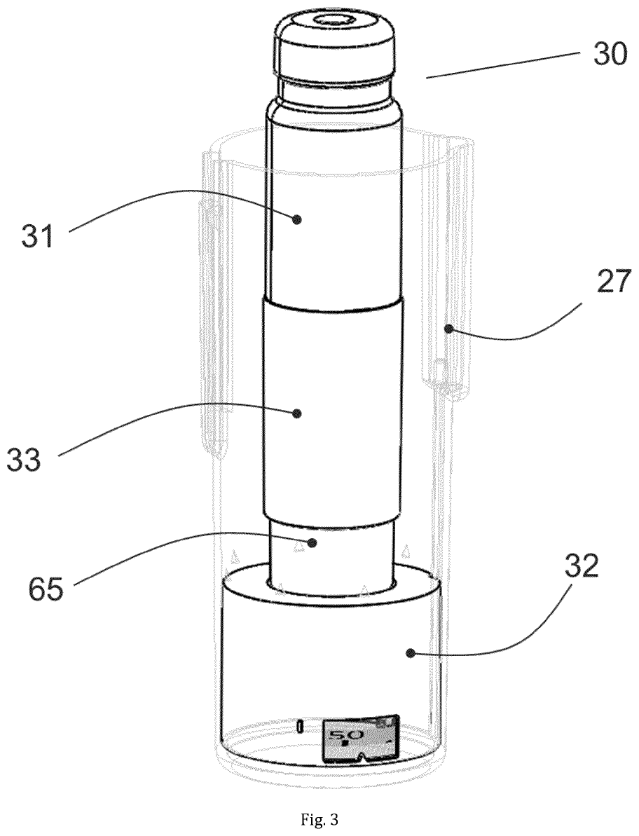

FIG. 3 shows a schematic view of the lower part of the housing (22) without the upper housing part. The counting unit (32) is located in the removable base (27) of the inhalation device system. The exchangeable cartridge system (30) comprises a container portion (31) and the blocking unit (33). The blocking unit (33) is physically separated from the counting unit (32) and comprises a counting member (65) which can interact with the counting unit.

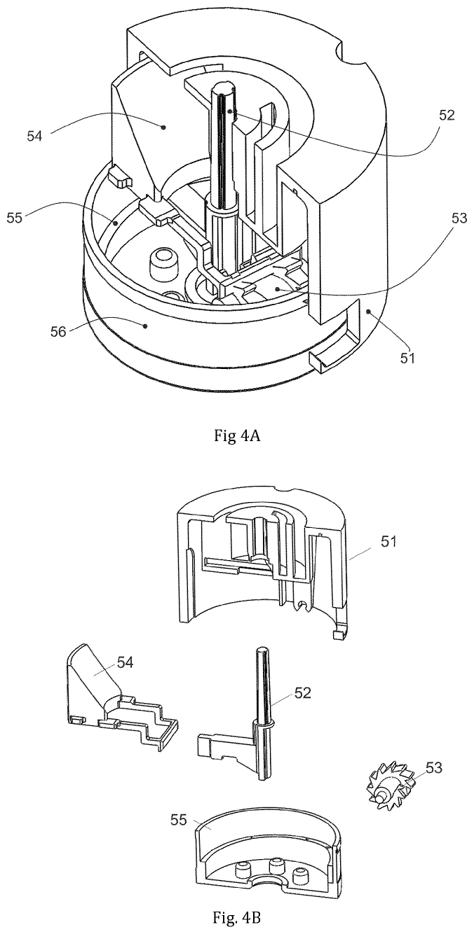

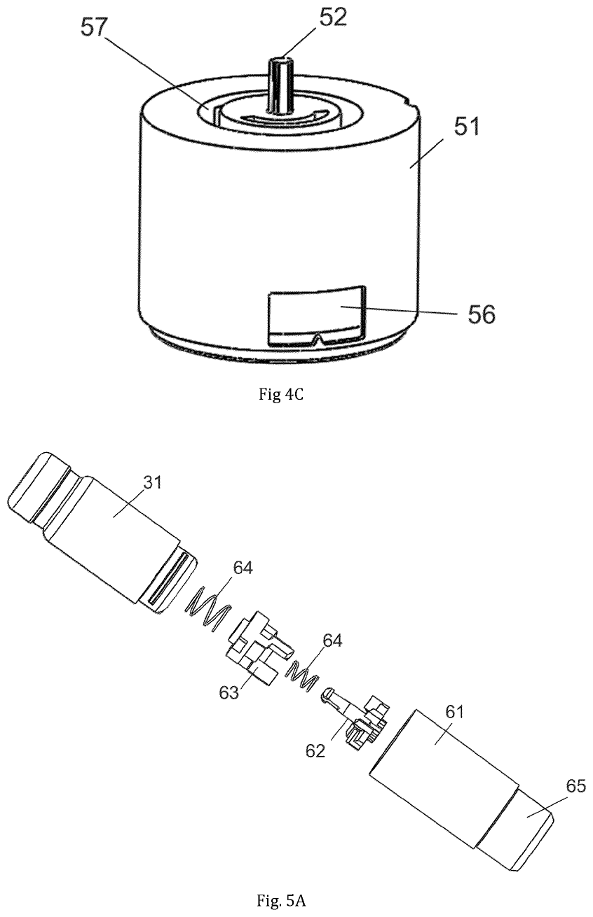

FIGS. 4A to 4C show an exemplary embodiment of a counting unit (32) according to the present invention. FIG. 4A shows a view inside the housing of the counting unit (51), showing the counting unit in assembled state. FIG. 4B shows a detailed view of the components of the counting unit and FIG. 4C shows the assembled counting unit without the inhaler device.

The counting unit (32) comprises a housing (51), in which a counting mechanism is located. The counting mechanism includes a moveable sleigh (54) which drives a counting gear (53) to move an indicator member (55) comprising face teeth and a sloped ramp (58). On the outer side of the indicator member (55) indexing means (56) are present to indicate the number of uses of the inhaler device system. The counting unit further comprises an activation member (52), moveable on the sloped ramp to increasing height, based on the position on the sloped ramp. The activation member adapted to interact with the blocking mechanism.

FIG. 5 shows an overview on an embodiment of a cartridge system according to the invention including a blocking unit according to the invention. FIG. 5A shows the container part (31) of the cartridge system, which can be connected to the housing of the blocking unit (61), which includes a counting member (65) for interacting with a counting unit. The blocking system comprises a plunger (62) and cam (63) which can interact and are held with a spring (64) mechanism.

FIG. 5B shows the inside of the housing of the blocking unit (62), which comprises protrusions (66), which limit the movement of the plunger (62) and cam (63) depending on the status of the blocking unit.

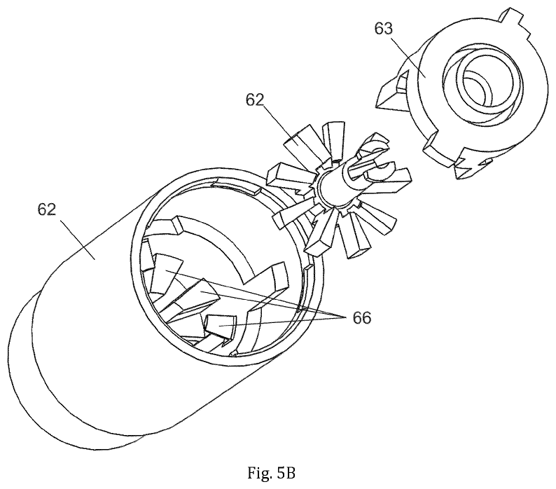

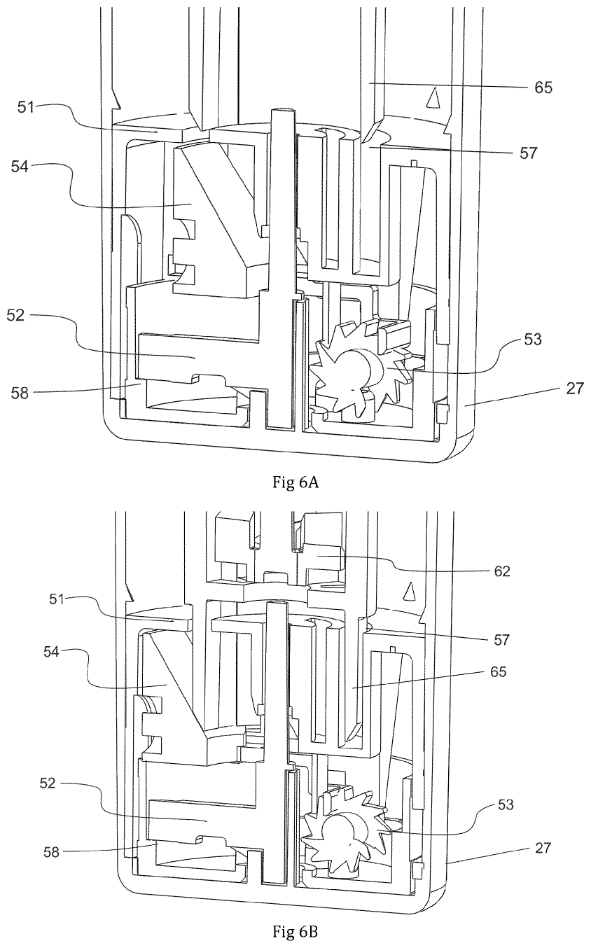

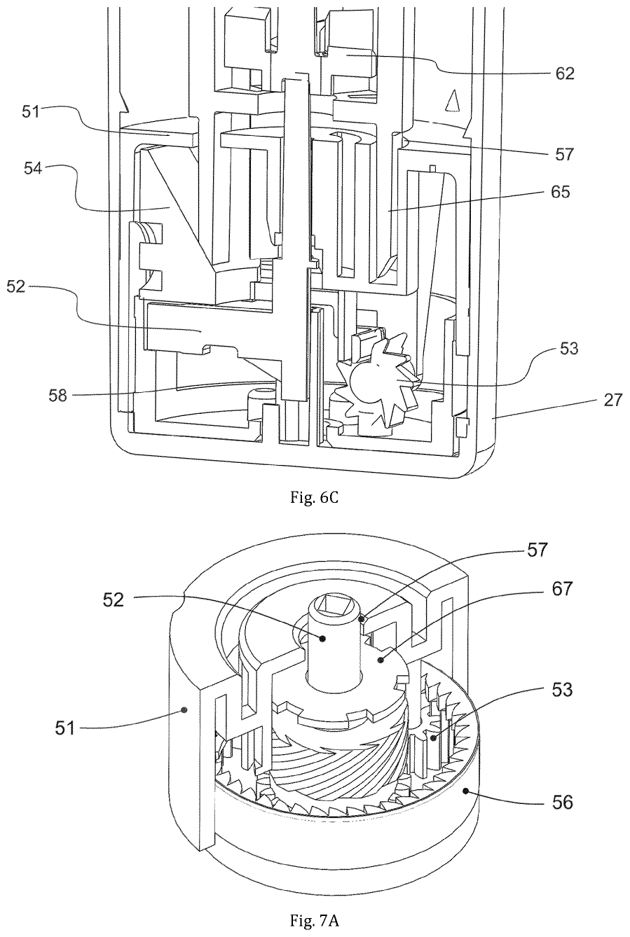

FIGS. 6A to 6C show an embodiment of the counting mechanism and a possible interaction of the blocking unit and the counting unit FIG. 6A shows the inhalation device system in the resting position. The counting member of the blocking system is above an opening of the of the counting unit and not interacting with the counting unit. The moveable sleigh (54) of the counting mechanism is in a neutral position. The position of the activation member (52) on the sloped ramp (58) of the indicator member (55) is dependent on the number uses before. FIG. 6A shows an early stage shortly after inserting a new exchangeable reservoir, wherein the activation member (62) is in a low position on the sloped ramp.

FIG. 6B shows the inhalation device system in the primed position. The counting member (65) of the blocking unit has entered the opening (57) of the counting unit, thereby moving the moveable sleigh (54). The movement of the moveable sleigh drives a counting gear (53), which in turn moves indicator member (55), causing the indexing means (56) to advance and the activation member (52) to move up on the sloped ramp. FIG. 6B further shows that the activation member does not interact with the blocking unit until the predefined number of uses is reached.

FIG. 6C shows the counting unit and blocking unit interacting after a predefined number of uses. The activation member (52) has moved on the sloped ramp (58) and is now able to interact with the blocking unit by interacting with the plunger (62).

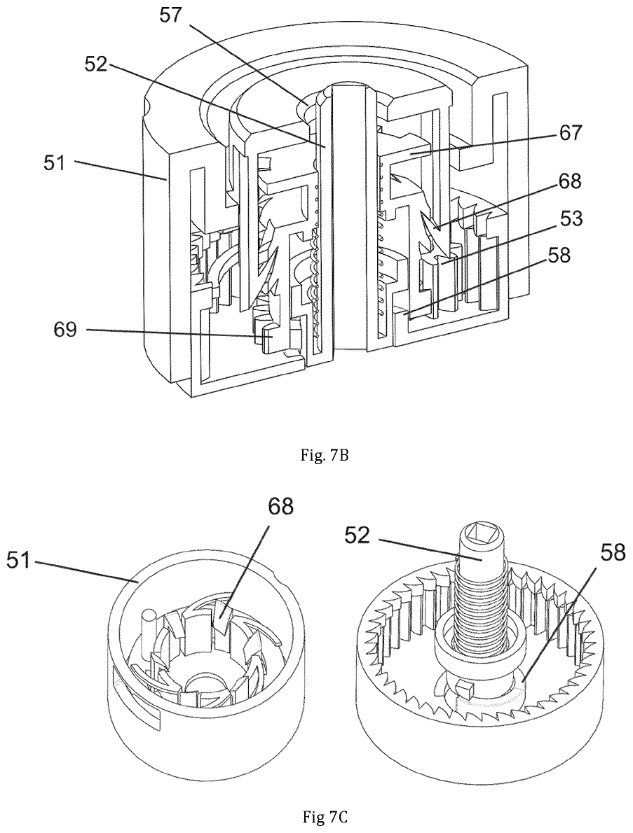

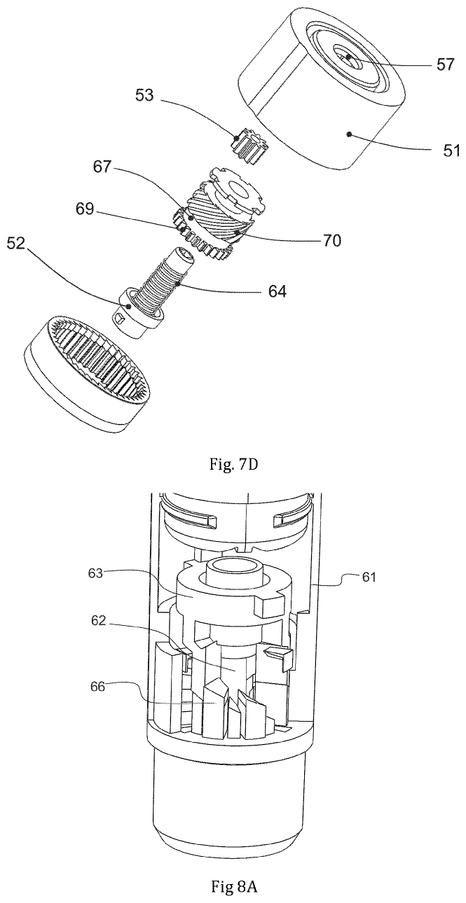

FIGS. 7A to 7D show an alternative embodiment of the counting mechanism. The counting mechanism comprises an activation member (52), which is placed on a sloped ramp (58) to interact with the clocking mechanism after a predetermined number of activations. FIG. 7A shows the counting mechanism through a cross section in the housing (51) and FIG. 7B shows a cross section of the counting mechanism. FIG. 7C chows the inner part of the housing (51) and the activation member (52) placed on the sloped ramp (58).

FIG. 7D shows the parts of the counting mechanism separately.

The alternative counting mechanism comprises a drive member (57) instead of a moveable sleigh (54). Said drive member comprises a helical guide threat (70) and radially arranged gear teeth (69). The helical guide threat (69) interacts with a guide member (68) arranged at the housing (51) of the counting unit. The drive member is moveable and is able to return to its original position with a spring (64).

In order to activate the counting unit, the counting member (55) of the blocking unit enters the opening (57) of the housing (51) and moves the drive member (67). The drive member is guided by the guide member (68) to move a gear (53) which in turn moves the indexing means (56), thereby also moving the activation member along the sloped ramp (58) until the activation member interacts with the blocking unit. It is also possible that the drive member in some embodiments moves the indexing means directly.

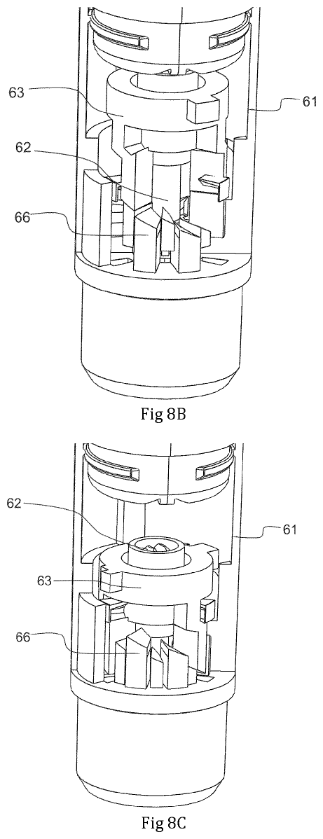

FIGS. 8A to 8C show an embodiment of the blocking mechanism. FIG. 8A shows the blocking mechanism in the initial position, in which the cam (63) is held in place by the protrusions (66) on the inside of the housing of the blocking unit (61). The plunger (62) is moveable inside the cam (63) and can be activated by the activation member (52) of the counting unit. Cam and plunger are held in place by springs (not shown).

FIGS. 8B and 8C show the activation of the blocking mechanism. After activation by the activation member (52) of the counting unit, the plunger (62) will move and activate the cam (63) moving the cam out of the protrusion (66) (FIG. 8B). Due to the spring mechanism (not shown) the cam (62) will move along the protrusion (66) into a blocking position (FIG. 8C) which prevents further movement of the plunger (62) and cam (63). In this case the activation member (52) of the counting unit prevents further movement of the cartridge, thus preventing further use of the cartridge.

### LIST OF REFERENCE NUMERALS

10 Inhalation device system
20 Inhalation device
21 housing
22 lower part of housing
23 receiving unit
24 connection unit
25 nozzle
26 cap
27 removable base
30 exchangeable cartridge system
31 container portion
32 counting unit
33 blocking unit
40 pumping unit
41 upstream end of the pumping unit
42 downstream end of the pumping unit
43 riser pipe
44 upstream end of the riser pipe
45 hollow cylinder of pumping unit
46 lockable means for storing potential energy
51 housing of the counting unit
52 activation member
53 gear
54 moveable sleigh
55 indicator member with sloped ramp and face teeth
56 indexing means
57 opening
58 sloped ramp
61 housing of the blocking unit
62 Plunger
63 Cam
64 Spring
65 Counting member
66 Protrusion
67 Drive member
68 Guide member
69 gear teeth
70 helical guide thread

1. Inhalation device system for the inhalative administration of a medically active liquid in nebulized form, the system comprising an inhalation device and an exchangeable reservoir for holding a plurality of doses of the medically active liquid, wherein one dose of the medically active liquid is dispensed from the inhalation device per actuation of the inhalation device system,
wherein the inhalation device comprises
a housing having a receiving unit, the receiving unit having a connection unit adapted to releasably and fluidically connect to a connection port of the exchangeable reservoir, the receiving unit being adapted to receive and fluidically connect to the exchangeable reservoir;
a nozzle for the nebulization of the medically active liquid; and
a pumping unit arranged within the housing and adapted to be fluidically connected to the exchangeable reservoir and to the nozzle and being adapted to convey the medically active liquid in a downstream direction from the exchangeable reservoir to the nozzle and being adapted to move the exchangeable reservoir from a resting position to a primed position upon priming of the pumping unit;
characterized in that
the inhalation device system comprises a combined counting and blocking assembly comprising a counting unit for counting the number of actuations of the inhalation device system following the insertion of the exchangeable reservoir into the inhalation device and a blocking unit for blocking the movement of the exchangeable reservoir from the resting position to the primed position when a defined number of actuations following the insertion of the exchangeable reservoir into the inhalation device is reached,
wherein the counting unit and the blocking unit are physically separated from each other when the exchangeable cartridge is in the resting position and adapted to physically interact with each other upon each movement of the exchangeable cartridge from the resting position to the primed position;
wherein either the blocking unit is attached to the exchangeable reservoir and the counting unit is attached to the housing of the inhalation device; or
the counting unit is attached to the exchangeable reservoir and the blocking unit is attached to the housing of the inhalation device.

2. Inhalation device system according to claim 1, wherein the counting unit is attached to the housing of the inhalation device and wherein the blocking unit is attached to the exchangeable reservoir.

3. Inhalation device system according to claim 1, wherein the exchangeable reservoir is in the form of an exchangeable cartridge having an upstream end and a downstream end and the blocking unit is attached to the upstream end of the exchangeable cartridge.

4. Inhalation device system according to claim 1, wherein the blocking unit has a housing containing a blocking mechanism.

5. Inhalation device system according to claim 4, wherein the housing of the blocking unit comprises an opening for interaction between the counting unit and the blocking mechanism.

6. Inhalation device system according to claim 4, wherein the counting unit comprises a counting mechanism and an activation mechanism.

7. Inhalation device system according to claim 6, wherein the activation mechanism interacts with the blocking mechanism of the blocking unit.

8. Inhalation device system according to claim 6, wherein the activation mechanism comprises an activation member for interaction with the blocking mechanism of the blocking unit; wherein said activation member is moveable and a position of the activation member changes with each actuation of the inhaler device system.

9. Inhalation device system according to claim 8, wherein the activation member is moving gradually or step-wise with each actuation.

10. Inhalation device system according to claim 8, wherein the activation member of the blocking unit enters the opening of the blocking unit for interaction with the blocking mechanism upon movement of the exchangeable reservoir from the resting position to the primed position.

11. Inhalation device system according to claim 6, wherein the counting mechanism is operated by interaction with the blocking unit upon movement of the exchangeable reservoir from the resting position to the primed position.

12. Inhalation device system according to claim 8, wherein the blocking mechanism limits the space for the activation member after a predefined number of uses so that movement of the exchangeable reservoir into the primed position is no longer possible.

13. An exchangeable cartridge system for the inhalation device system comprising the combined counting and blocking assembly according to claim 1 for holding the medically active liquid for nebulization and adapted for use in the inhalation device system according to items claim 1, wherein the cartridge system comprises a container portion having an effective volume Vₑ for holding the medically active liquid and a connection port adapted to releasably and fluidically connect the cartridge system to the pumping unit, specifically via the connection unit of the receiving unit of the inhalation device, wherein the cartridge system comprises the blocking unit adapted to interact with the counting unit of the inhalation device system comprising the combined counting and blocking assembly according to claim 1.

14. An inhalation device for the inhalation device system comprising the combined counting and blocking assembly according to claim 1,
the counting unit adapted to interact with the blocking unit of the combined counting and blocking assembly according to claim 1; and/or
the blocking unit, adapted to interact with the counting unit of the combined counting and blocking assembly according to claim 1.
